(19) United States
(12) Patent Application Publication
(10) Pub. No.: US 2021/0393588 A1
(43) Pub. Date: Dec. 23, 2021

(54) READY TO USE INTRAVENOUS INFUSION OF BRIVARACETAM OR SALT THEREOF

(71) Applicant: Zenvision Pharma LLP, Mumbai (MH)

(72) Inventors: Sivakumar Venkata Bobba, Navi Mumbai (IN); Bhimrao Jadhav, Navi Mumbai (IN); Dhananjay Shinde, Navi Mumbai (IN); Sunil Pophale, Navi Mumbai (IN)

(21) Appl. No.: 17/289,786

(22) PCT Filed: Oct. 31, 2019
(86) PCT No.: PCT/IB2019/059347

§ 371 Date: Apr. 29, 2021

(30) Foreign Application Priority Data
Nov. 2, 2018 (IN) 201821041531

(51) Int. Cl. A61K 31/4015; A61K 9/00; A61K 47/02; A61K 47/12

(52) U.S. Cl. CPC A61K 31/4015; A61K 47/12; A61K 47/02; A61K 9/0019

(57) ABSTRACT

The present disclosure relates to the ready to use intravenous infusion of Brivaracetam or salt thereof. The ready to use intravenous infusion of Brivaracetam or salt thereof provides longer stability, which can be administered for longer period of time, with direct use without prior dilution, which saves time for dilution in case of emergency, with better ease of handling and which is efficacious; provide better patient compliance in the treatment of seizures.

### FIELD OF THE INVENTION

The present invention relates to ready to use intravenous infusion of brivaracetam or salt thereof for the treatment of seizures.

### BACKGROUND OF THE INVENTION

Brivaracetam is a third-generation antiepileptic racetam derivative and a 4-n-propyl analogue of levetiracetam. Chemically brivaracetam is (2S)-2-[(4R)-2-oxo-4-propyltetrahydro-1H-pyrrol-1-yl]butanamide and its molecular weight is 212.29. Its empirical formula is C₁₁H₂₀N₂O₂. Brivaracetam is represented by compound of structural formula I

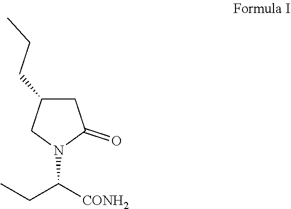

Brivaracetam is a white to off-white crystalline powder. It is very soluble in water, buffer (pH 1.2, 4.5, and 7.4), ethanol, methanol, and glacial acetic acid. It is freely soluble in acetonitrile and acetone and soluble in toluene. It is very slightly soluble in n-hexane.

Brivaracetam intravenous solution of UCB INC has been approved in USA as on May 12, 2016 under the trade name BRIVIACT® and is available in the strength of 50 mg/5 ml (10 mg/ml). The product is indicated for the treatment of partial-onset seizures only in adult patients.

The commercially available Brivaracetam intravenous solution vial contains 50 mg of brivaracetam and sodium acetate (trihydrate), glacial acetic acid, sodium chloride and water for injection as inactive ingredients.

The USFDA product label for brivaracetam injection discloses it can be administered intravenously without further dilution or may be mixed with diluents like 0.9% Sodium Chloride injection USP, Lactated Ringer's injection, 5% Dextrose injection, USP. It also further discloses diluted solution should not be stored for more than 4 hours at room temperature and may be stored in polyvinyl chloride (PVC) bags. The said product label does not discloses ready to use intravenous brivaracetam infusion as well as long term stability of intravenous brivaracetam infusion.

The public assessment report for the brivaracetam injection of European medicine agency discloses “Stability of the brivaracetam injection in different perfusion diluents (0.9% saline solution, 5% glucose solution, lactated Ringer's solution) at various concentrations and in either PVC or polyolefin perfusion bags was tested. The product is stable for up to 72 hours under such conditions. The said public assessment report does not discloses ready to use intravenous brivaracetam infusion as well as long term stability of intravenous brivaracetam infusion.

Commercially available product and product known in the prior art for brivaracetam or salt thereof are available in the form of tablet, oral solution, injection. The product known in the prior art for brivaracetam injection requires manual dilution with diluents like sodium chloride, lactated ringers, dextrose to prepare infusion. The said manually prepared infusions are stable for 4 to 72 hours and require immediate administration. The said manually prepared infusion cannot be stored for long duration.

Thus there is an unmet need in the art to provide ready to use intravenous infusion of brivaracetam or salt thereof with longer stability, which can be administered for longer period of time at optimum rate to obtain desired therapeutic effect, which can be used directly without prior dilution, which saves time for dilution in case of emergency, with better ease of handling to the medical professional and which is effective and provides better patient compliance in the treatment of seizures.

Accordingly, applicant of the present invention invented ready to use intravenous infusion of brivaracetam or salt thereof with longer stability, which can be administered for longer period of time, with direct use without prior dilution, which saves time for dilution in case of emergency, with better ease of handling and which is efficacious, provide better patient compliance in the treatment of seizures.

### OBJECTS OF THE INVENTION

Accordingly it is an object of the present invention to provide ready to use infusion of Brivaracetam or salt thereof.

It is an object of the present invention to provide ready to use intravenous infusion of Brivaracetam or salt thereof.

It is another object of the present invention to provide a ready to use intravenous infusion composition comprising Brivaracetam or salt thereof, an antioxidant, a vehicle and one or more pharmaceutically acceptable excipients.

It is another object of the present invention to provide ready to use intravenous infusion of Brivaracetam or salt thereof which provides longer stability.

It is another object of the present invention to provide ready to use intravenous infusion of Brivaracetam or salt thereof comprising antioxidant which provides longer stability.

It is another object of the present invention to provide ready to use intravenous infusion of Brivaracetam or salt thereof which pH ranges from 3 to 7 and provides longer stability.

It is another object of the present invention to provide stable ready to use intravenous infusion of Brivaracetam or salt thereof which can be administered for longer period of time.

It is another object of the present invention to provide stable ready to use intravenous infusion of Brivaracetam or salt thereof with direct use without prior dilution, which saves time for dilution in case of emergency.

It is another object of the present invention to provide stable ready to use intravenous infusion of Brivaracetam or salt thereof which is efficacious and provides better ease of handling and better patient compliance in the treatment of seizures.

### SUMMARY OF THE INVENTION

A first aspect of the present invention is to provide ready to use infusion of Brivaracetam or salt thereof.

In another aspect of the present invention is to provide ready to use intravenous infusion of Brivaracetam or salt thereof.

In another aspect of the present invention is to provide stable ready to use intravenous infusion of Brivaracetam or salt thereof.

In another aspect of the present invention is to provide a ready to use intravenous infusion composition comprising Brivaracetam or salt thereof, an antioxidant, a vehicle and one or more pharmaceutically acceptable excipients.

In another aspect of the present invention is to provide stable ready to use intravenous infusion of Brivaracetam or salt thereof comprising antioxidant along with one or more pharmaceutically acceptable excipient.

In another aspect of the present invention is to provide stable ready to use intravenous infusion of Brivaracetam or salt thereof along with one or more pharmaceutically acceptable excipient with pH ranges from 3 to 7.

In another aspect of the present invention is to provide stable ready to use intravenous infusion of Brivaracetam or salt thereof along with sodium chloride or lactated ringers or dextrose or mixture thereof; optionally along with one or more pharmaceutically acceptable excipient.

In another aspect of the present invention is to provide process of manufacturing ready to use intravenous infusion of Brivaracetam or salt thereof along with one or more pharmaceutically acceptable excipient.

In another aspect of the present invention is to provide process of manufacturing ready to use intravenous infusion of Brivaracetam or salt thereof along with sodium chloride or lactated ringers or dextrose or mixture thereof; optionally along with one or more pharmaceutically acceptable excipient.

In another aspect of the present invention is to provide method of treating seizures by ready to use intravenous infusion of Brivaracetam or salt thereof.

### DETAIL DESCRIPTION OF THE INVENTION

The present invention relates to ready to use intravenous infusion of Brivaracetam or salt thereof.

The present invention relates to stable ready to use intravenous infusion of Brivaracetam or salt thereof.

The term stable or stability according to present invention means the ability of the pharmaceutical dosage form to maintain the physical, chemical, therapeutic and microbial properties during the time of storage and usage by the patient.

Aqueous solutions of Brivaracetam were found to be partially unstable during the storage. Also degradation products in solution are formed due to oxidation, pH ranges.

Accordingly, the present invention provide stable ready to use intravenous infusion of Brivaracetam or salt thereof comprising antioxidant along with one or more pharmaceutically acceptable excipient.

The present invention also provide stable ready to use intravenous infusion of Brivaracetam or salt thereof along with one or more pharmaceutically acceptable excipient which pH ranges from 3 to 7.

The pharmaceutical composition of the present invention in the form ready to use infusion can comprise any suitable amount of the Brivaracetam or salts thereof. The weight percentage of Brivaracetam ranges from 0.001% to 20%, preferably 0.01% to 10% based on the total volume of composition.

The stable ready to use intravenous infusion of Brivaracetam or salt thereof according to present invention may contain one or more pharmaceutically acceptable excipient.

The stable ready to use intravenous infusion of Brivaracetam or salt thereof according to present invention may contain sodium chloride or lactated ringers or dextrose or mixture thereof optionally with one or more pharmaceutically acceptable excipient.

The one or more pharmaceutically acceptable excipient according to present invention may be selected from the group consisting of antioxidants, osmotic or tonicity adjusting agents, chelating agents, pH adjusting agents, buffers, preservatives, surfactants, solvents/co-solvents and vehicles.

The examples of antioxidants include but not limited to ascorbic acid, sodium metabisulfite, sodium bisulfite, sodium formaldehyde sulfoxylate, thiourea, butylated hydroxyl anisole, butylated hydroxytoluene, ascorbic acid esters, propyl gallate, vitamin E, alpha-tocopherol or combinations thereof. The amount of antioxidant present in the composition ranges from about 0.001% to 10%; preferably about 0.001% to 5%; more preferably about 0.001 to 2% based on the total volume of the composition.

The examples of osmotic or tonicity adjusting agents include but not limited to sodium chloride, potassium chloride, calcium chloride, mannitol, glycerol, sorbitol, propylene glycol, dextrose, sucrose or combinations thereof. The amount of osmotic or tonicity adjusting agents present in the composition ranges from about 0.001% to 10%; preferably about 0.01 to 5% based on the total volume of the composition.

The examples of chelating agents include but not limited to disodium edetate dihydrate, disodium edetate, edetic acid, ethylenediamine tertaacetic acid, calcium disodium ethylenediamine tertaacetic acid, diethylenetriamine pentaacetic acid, calcium versetamide sodium or combinations thereof.

The examples of pH adjusting agents according to present invention include but not limited to hydrochloric acid, citric acid, sulfuric acid, acetic acid, tartaric acid, lactic acid, tromethamine, sodium hydroxide, potassium hydroxide, sodium carbonate or combinations thereof. The pH of the ready to use infusion may ranges from 3 to 7.

The examples of buffers include but not limited Sodium acetate trihydrate, phosphate, citrate, tris, succinate, histidine, glycine, arginine, malic, tartaric, acetic, benzoic, gluconic, glyceric, lactic, aconitic, adipic, ascorbic, carbonic, glutamic, ammonium chloride, triethanolamine or combinations thereof. The amount of buffering agent present in the composition ranges from about 0.001% to 10%; preferably about 0.01% to 5% based on the total volume of the composition.

The examples of preservative include but not limited to parabens, benzalkonium chloride, benzethonium chloride, benzyl alcohol, chlorobutanol, m-cresol, myristyl gamma picolinium chloride, phenol, 2-phenoxyethanol, phenyl mercuric nitrate, phenyl ethyl alcohol, EDTA or combinations thereof.

The examples of surfactants include but not limited to Polyoxyethylene sorbitan monooleate (Tween 80), Sorbitan monooleate, Polyoxyethylene sorbitan monolaurate (Tween 20), Lecithin, Polyoxyethylene polyoxypropylene copolymers (Pluronics) or combinations thereof.

The examples of solvents/co-solvents include but not limited to benzyl benzoate, ethyl oleate, isopropyl myristate, castor oil, cotton seed oil, N,N-dimethylacetamide, ethanol, ethanol dehydrated, dioxolanes, N-methyl 2-pyrrolidone, polyethylene glycol, propylene glycol, butylene glycol, glycerin, sesame oil, soybean oil, vegetable oil or combinations thereof.

The examples of vehicles include but not limited to sodium chloride, lactated ringers, dextrose, water for injection, sterile water for injection, bacteriostatic water for injection, water miscible solvents like dioxolanes, dimethylacetamide, N-(β-hydroxyethyl)-lactamide, butylene glycol, polyethylene glycol, propylene glycol, glycerin, ethyl alcohol, water immiscible solvents like ethyl oleate, isopropyl myristate, benzyl benzoate, fixed oil, corn oil, cottonseed oil, peanut oil, sesame oil or combinations thereof.

In another aspect of the present invention is to provide process of manufacturing ready to use intravenous infusion of brivaracetam or salt thereof along with one or more pharmaceutically acceptable excipients.

The process of manufacturing ready to use intravenous infusion of brivaracetam or salt involves steps of
i) Preparation of brivaracetam injection.
ii) Diluting the prepared brivaracetam injection of the step 1 into the vehicle like sodium chloride or lactated ringers or dextrose or mixture thereof to form brivaracetam infusion.
iii) Adjusting the pH in the range of 3 to 7.
iv) Packaging of the prepared brivaracetam infusion of the step 2 into the bags.

The process of manufacturing ready to use intravenous infusion of brivaracetam preferably involves steps of
i) Preparation of brivaracetam injection:
   Dissolving Brivaracetam and one or more pharmaceutically acceptable excipients like buffers, tonicity adjusting agents and pH adjusting agent optionally with chelating agents, antioxidants, preservatives, surfactants, solvents/co-solvents into the vehicle; Filter the said Brivaracetam injection through 0.2 micron PES filter.
ii) Preparation of brivaracetam infusion:
   Mixing or diluting the prepared brivaracetam injection into the vehicle like sodium chloride or lactated ringers or dextrose or mixture thereof to form brivaracetam infusion. The said brivaracetam infusions were packaged into the bags.

The concentration of Brivaracetam or salt thereof, antioxidant, sodium chloride or lactated ringers or dextrose or mixture thereof, excipients, pH ranges and manufacturing process has been optimized in such way that stable ready to use intravenous infusion of brivaracetam or salt thereof according to present invention provide longer stability, can be administered for longer period of time, direct use without prior dilution, saves time for dilution in case of emergency, better ease of handling, efficacious and provide better patient compliance in the treatment of seizures.

The pharmaceutical compositions of the present invention were evaluated initially and after stability studies for the parameters like appearance, pH, osmolarity, sterility, assay, leakage test, pyrogen test, volume filled, clarity test, particulate matter test and found to be in the compliance. Therefore brivaracetam infusions according to present invention were found to be physically, chemically and therapeutically stable during the time of storage. Preferably brivaracetam infusions according to present invention were found to be stable for more than 72 hours.

The stable ready to use intravenous infusion of brivaracetam or salt thereof according to present invention can be packaged in suitable flexible infusion container which is made up of a plastic material or other polymeric material or glass container. The stable ready to use intravenous infusion of brivaracetam or salt thereof according to present invention can be packaged in the flexible infusion container like an infusion bag, a flexible infusion pouch, a soft bag, an infusion bottle.

### EXAMPLES

The following Examples are provided solely for illustrative purposes and are not meant to limit the invention in any way.

### Example 1

| | | | |
|---|---|---|---|
| Sr. No. | Ingredients | mg/ml | mg/5 ml |

Part A: Brivaracetam injection

| | | | |
|---|---|---|---|
| 1. | Brivaracetam | 10.00 | 50.00 |
| 2. | Sodium chloride | 9.0 | 45.00 |
| 3. | Sodium acetate trihydrate | 1.64 | 8.20 |
| 4. | Water for injection | QS | QS |
| 5. | Acetic acid (0.1M) | QS | QS |

| | | |
|---|---|---|
| Total weight | 1.00 | 5.00 |

| | | |
|---|---|---|
| Sr. No. | Ingredients | Quantity |

Part B: Brivaracetam infusion

| | | |
|---|---|---|
| 1. | Brivaracetam injection | 5.00 ml |
| | (Part A) | |
| 2. | Sodium chloride | 0.9% |
| 3. | Water for injection | QS |

| | |
|---|---|
| Total weight | 100.00 ml |

### Manufacturing Process:

### Part A: Brivaracetam Injection

1. Dissolve required quantity of sodium chloride to sterile water for injection and dissolve completely.
2. Further dissolve sodium acetate trihydrate to sodium chloride solution (0.9% w/v), obtained in step 1 and dissolve completely.
3. Add 50.00 mg of Brivaracetam to solution of step 2 and dissolve.
4. Adjust the pH of the solution obtained in step 3, to 3 using 0.1M acetic acid solution.
5. Adjust the volume to 5.00 ml using sodium chloride solution (0.9% w/v). 6. Filter using 0.2 micron PES filter.

### Part B: Brivaracetam Infusion

1. Dissolve required quantity of sodium chloride to sterile water for injection and dissolve completely to obtain sodium chloride solution (0.9% w/v).
2. Pipette out 5.00 ml of Brivaracetam injection obtained in Part A and mix with 95.00 ml sterile sodium chloride solution (0.9% w/v).
   Evaluation Parameters after Five Days Stability Study (40° C./75 RH):

| Sr. No. | Parameters | Observations |
|---|---|---|
| 1. | Appearance | Clear, transparent, homogenous solution |
| 2. | pH | 3.51 |
| 3. | Assay | 104.0% |
| 4. | Osmolarity | 319 mOsm/Kg |

### Example 2

| | | | |
|---|---|---|---|
| Sr. No. | Ingredients | mg/ml | mg/5 ml |

Part A: Brivaracetam injection

| | | | |
|---|---|---|---|
| 1. | Brivaracetam | 10.00 | 50.00 |
| 2. | Sodium chloride | 9.0 | 45.0 |
| 3. | Sodium acetate trihydrate | 1.64 | 8.20 |
| 4. | Ascorbic acid | 6.6 | 0.66 |
| 5. | Water for injection | QS | QS |
| 6. | Acetic acid (0.1M) | QS | QS |

| | | |
|---|---|---|
| Total weight | 1.00 | 5.00 |

| | | |
|---|---|---|
| Sr. No. | Ingredients | Quantity |

Part B: Brivaracetam infusion

| | | |
|---|---|---|
| 1. | Brivaracetam injection | 5.00 ml |
| | (Part A) | |
| 2. | Sodium chloride | 0.9% |
| 3. | Water for injection | QS |

| | |
|---|---|
| Total weight | 100.00 ml |

### Manufacturing Process:

### Part A: Brivaracetam Injection

1. Dissolve required quantity of sodium chloride to sterile water for injection and dissolve completely.
2. Further dissolve sodium acetate trihydrate and ascorbic acid to sodium chloride solution (0.9% w/v), obtained in step 1 and dissolve completely.
3. Add 50.00 mg of Brivaracetam to solution of step 2 and dissolve.
4. Adjust the pH of the solution obtained in step 3, to 3.2 using 0.1M acetic acid solution.
5. Adjust the volume to 5.00 ml using sodium chloride solution (0.9% w/v).
6. Filter using 0.2 micron PES filter.

### Part B: Brivaracetam Infusion

1. Dissolve required quantity of sodium chloride to sterile water for injection and dissolve completely to obtain sodium chloride solution (0.9% w/v).
2. Pipette out 5.00 ml of Brivaracetam injection obtained in Part A and mix with 95.00 ml sterile sodium chloride solution (0.9% w/v).
   Evaluation Parameters after Five Days Stability Study (40° C./75 RH):

| Sr. No. | Parameters | Observations |
|---|---|---|
| 1. | Appearance | Clear, transparent, homogenous solution |
| 2. | pH | 3.01 |
| 3. | Assay | 103.6% |
| 4. | Osmolarity | 349 mOsm/kg |

### Example 3

| | | | |
|---|---|---|---|
| Sr. No. | Ingredients | mg/ml | mg/5 ml |

Part A: Brivaracetam injection

| | | | |
|---|---|---|---|
| 1. | Brivaracetam | 10.00 | 50.00 |
| 2. | Sodium chloride | 9.0 | 45.0 |
| 3. | Sodium acetate trihydrate | 1.64 | 8.20 |
| 4. | Water for injection | QS | QS |
| 5. | Sodium hydroxide (0.1M) | QS | QS |

| | | |
|---|---|---|
| Total weight | 1.00 | 5.00 |

| | | |
|---|---|---|
| Sr. No. | Ingredients | Quantity |

Part B: Brivaracetam infusion

| | | |
|---|---|---|
| 1. | Brivaracetam injection | 5.00 ml |
| | (Part A) | |
| 2. | Sodium chloride | 0.9% |
| 3. | Water for injection | QS |

| | |
|---|---|
| Total weight | 100.00 ml |

### Manufacturing Process:

### Part A: Brivaracetam Injection

1. Dissolve required quantity of sodium chloride to sterile water for injection and dissolve completely.
2. Further dissolve sodium acetate trihydrate to sodium chloride solution (0.9% w/v), obtained in step 1 and dissolve completely.
3. Add 50.00 mg of Brivaracetam to solution of step 2 and dissolve.
4. Adjust the pH of the solution obtained in step 3, to 7.0 using 0.1M sodium hydroxide solution.
5. Adjust the volume to 5.00 ml using sodium chloride solution (0.9% w/v).
6. Filter using 0.2 micron PES filter.

### Part B: Brivaracetam Infusion

1. Dissolve required quantity of sodium chloride to sterile water for injection and dissolve completely to obtain sodium chloride solution (0.9% w/v).
2. Pipette out 5.00 ml of Brivaracetam injection obtained in Part A and mix with 95.00 ml sterile sodium chloride solution (0.9% w/v).
   Evaluation Parameters after Five Days Stability Study (40° C./75 RH):

| Sr. No. | Parameters | Observations |
|---|---|---|
| 1. | Appearance | Clear, transparent, homogenous solution |
| 2. | pH | 7.14 |
| 3. | Assay | 100.9% |
| 4. | Osmolarity | 311 mOsm/kg |

### Example 4

| | | | |
|---|---|---|---|
| Sr. No. | Ingredients | mg/ml | mg/5 ml |

Part A: Brivaracetam injection

| | | | |
|---|---|---|---|
| 1. | Brivaracetam | 10.00 | 50.00 |
| 2. | Sodium chloride | 9.00 | 45.0 |
| 3. | Sodium acetate trihydrate | 1.64 | 8.20 |
| 4. | Sodium metabisulphite | 2.00 | 0.20 |
| 5. | Water for injection | QS | QS |
| 6. | Sodium hydroxide (0.1M) | QS | QS |

| | | |
|---|---|---|
| Total weight | 1.00 | 5.00 |

| | | |
|---|---|---|
| Sr. No. | Ingredients | Quantity |

Part B: Brivaracetam infusion

| | | |
|---|---|---|
| 1. | Brivaracetam injection | 5.00 ml |
| | (Part A) | |
| 2. | Sodium chloride | 0.9% |
| 3. | Water for injection | QS |

| | |
|---|---|
| Total weight | 100.00 ml |

### Manufacturing Process:

### Part A: Brivaracetam Injection

1. Dissolve required quantity of sodium chloride to sterile water for injection and dissolve completely.
2. Further dissolve sodium acetate trihydrate and sodium metabisulphite to sodium chloride solution (0.9% w/v), obtained in step 1 and dissolve completely.
3. Add 50.00 mg of Brivaracetam to solution of step 2 and dissolve.
4. Adjust the pH of the solution obtained in step 3, to 6.8 using 0.1M sodium hydroxide solution.
5. Adjust the volume to 5.00 ml using sodium chloride solution (0.9% w/v).
6. Filter using 0.2 micron PES filter.

### Part B: Brivaracetam Infusion

1. Dissolve required quantity of sodium chloride to sterile water for injection and dissolve completely to obtain sodium chloride solution (0.9% w/v).
2. Pipette out 5.00 ml of Brivaracetam injection obtained in Part A and mix with 95.00 ml sterile sodium chloride solution (0.9% w/v).
   Evaluation Parameters after Five Days Stability Study (40° C./75 RH):

| Sr. No. | Parameters | Observations |
|---|---|---|
| 1. | Appearance | Clear, transparent, homogenous solution |
| 2. | pH | 7.0 |
| 3. | Assay | 106.3% |
| 4. | Osmolarity | 331 mOsm/kg |

1. A ready to use infusion of Brivaracetam or salt thereof.

2. The ready to use infusion of Brivaracetam or salt thereof according to claim 1 comprising one or more antioxidants selected from ascorbic acid, sodium metabisulfite, sodium bisulfite, sodium formaldehyde sulfoxylate, thiourea, butylated hydroxyl anisole, butylated hydroxytoluene, ascorbic acid esters, propyl gallate, vitamin E, alpha-tocopherol or combinations thereof.

3. The ready to use infusion of Brivaracetam or salt thereof according to claim 1 is stable.

4. The ready to use infusion of Brivaracetam or salt thereof according to claim 1, with pH ranges from 3 to 7.

5. The ready to use infusion of Brivaracetam or salt thereof according to claim 1 is stable for more than 72 hours.

6. The infusion according to claim 2 further comprises vehicles selected from sodium chloride or lactated ringers or dextrose, water for injection, sterile water for injection, bacteriostatic water for injection, water miscible solvents like dioxolanes, dimethylacetamide, N-(β-hydroxyethyl)-lactamide, butylene glycol, polyethylene glycol, propylene glycol, glycerin, ethyl alcohol, water immiscible solvents like ethyl oleate, isopropyl myristate, benzyl benzoate, fixed oil, corn oil, cottonseed oil, peanut oil, sesame oil or combinations thereof.

7. The infusion according to claim 6, further comprises one or more pharmaceutically acceptable excipients selected from the group consisting of osmotic or tonicity adjusting agents, chelating agents, pH adjusting agents, buffers, preservatives, surfactants, solvents/co-solvents.

8. The infusion according to claim 7 preferably comprises osmotic or tonicity adjusting agents, pH adjusting agents and buffers

9. The infusion according to claim 8, wherein osmotic or tonicity adjusting agents is selected from the group consisting of sodium chloride, potassium chloride, calcium chloride, mannitol, glycerol, sorbitol, propylene glycol, dextrose, sucrose or combinations thereof; pH adjusting agents is selected from the group consisting of hydrochloric acid, citric acid, sulfuric acid, acetic acid, tartaric acid, lactic acid, tromethamine, sodium hydroxide, potassium hydroxide, sodium carbonate or combinations thereof and buffers selected from the group consisting of Sodium acetate trihydrate, phosphate, citrate, tris, succinate, histidine, glycine, arginine, malic, tartaric, acetic, benzoic, gluconic, glyceric, lactic, aconitic, adipic, ascorbic, carbonic, glutamic, ammonium chloride, triethanolamine or combinations thereof.

10. The process of manufacturing ready to use infusion of Brivaracetam or salt thereof involves steps of
i) Preparation of brivaracetam injection.
ii) Diluting the prepared brivaracetam injection of the step 1 into the vehicle like sodium chloride or lactated ringers or dextrose or mixture thereof to form brivaracetam infusion.
iii) Adjusting the pH in the range of 3 to 7.
iv) Packaging of the prepared brivaracetam infusion of the step 2 into the bags.

11. A ready to use intravenous infusion composition comprising Brivaracetam or salt thereof, an antioxidant, a vehicle and one or more pharmaceutically acceptable excipients.

12. The infusion according to claim 11, further comprises one or more pharmaceutically acceptable excipients selected from the group consisting of osmotic or tonicity adjusting agents, chelating agents, pH adjusting agents, buffers, preservatives, surfactants, solvents/co-solvents.

13. A ready to use intravenous infusion composition of Brivaracetam or salt thereof according to claim 1, for use in the treatment of partial-onset seizures.
